(19) United States
(12) Patent Application Publication
(10) Pub. No.: US 2019/0216733 A1
(43) Pub. Date: Jul. 18, 2019

(54) COLD-PROCESSED SELF-EMULSIFYING HYDROALCOHOLIC GEL COMPOSITIONS AND METHODS

(71) Applicant: Avaria Health and Beauty Corp., Cambridge (CA)

(72) Inventors: Keith Burk, Cambridge (CA); Philip Hudson, Cambridge (CA); Fan Wang, Cambridge (CA)

(73) Assignee: Avaria Health and Beauty Corp., Cambridge (CA)

(21) Appl. No.: 16/250,884

(22) Filed: Jan. 17, 2019

(30) Foreign Application Priority Data
Jan. 17, 2018 (CA) 2992201

(51) Int. Cl. A61K 9/06; A61K 9/00; A61K 47/10; A61K 47/20; A61K 47/26; A61K 47/12; A61K 31/352; A61K 31/455

(52) U.S. Cl. CPC A61K 9/06; A61K 9/0014; A61K 47/10; A61K 31/455; A61K 47/26; A61K 47/12; A61K 31/352; A61K 47/20

(57) ABSTRACT

A hydroalcoholic gel acts as a carrier system for bioactive substrates. This hydroalcoholic gel contains water and lower alcohol as the continuous phase. It also contains gelling agents, thickening agents, skin conditioning agents, emulsifiers, and penetration enhancers. This hydroalcoholic gel can be manufactured through a process that does not require external heating. This hydroalcoholic gel can self-emulsify into a lotion or cream texture when an oil is introduced to the system. Bioactive substrates that can be incorporated into this carrier hydroalcoholic gel can be hydrophilic or hydrophobic. Hydrophilic bioactive substrates can be dispersed into the gel directly, while hydrophobic substrates can be incorporated firstly into an oil and then into the carrier gel through a self-emulsification process. This hydroalcoholic gel can enhance the topical delivery efficacy of certain substrates.

### TECHNICAL FIELD

The present disclosure relates to the field of drug delivery. In particular, the present disclosure relates to compositions and methods of use thereof for the topical delivery of biological actives, including pharmaceutical actives, through the skin and/or mucus membranes in humans and animals.

### BACKGROUND

The compositions of the present disclosure are useful as carriers for topical application of drugs, pharmaceutical ingredients, and cosmeceutical active ingredients. The common knowledge of cutaneous delivery suggests that the bioavailability of topical drugs is very low. The skin is a hydrophobic barrier and the topical delivery efficacy of hydrophilic substrates is therefore low through intact skin. Therefore, a topical carrier gel should contain penetration enhancers, which could help disrupt the skin barrier and deliver active components. Hydrophobic substrates, such as steroids, terpenoids, amides, and some plant extracts or essential oils are subjected to degradation when water is present. When used as topical drugs, pharmaceuticals, or cosmetic actives, these substrates normally come in existing formulations or as bulk chemicals which need to be dispersed into a carrier base in compounding practice. On-site dispersion of these hydrophobic (or lipophilic) substrates into a carrier base enhances their stability. Creams or gels are the typical forms of carriers.

One of the difficulties of current methods of dispersing lipophilic substrates into an opaque carrier base is to ensure uniform dispersion, where shear mixers or electro-mortars are commonly used in a compounding pharmacy to ensure homogeneity. Some traditional methods of effective topical delivery of certain substrates, such as cannabinols including Δ-9 tetrahydrocannabinol (THC) and cannabidiol (CBD) require an occlusive body such as a patch. The compositions of the present disclosure overcome the requirement for use of an occlusive system for topical delivery of certain bioactive substrates using a non-occlusive topical gel or its emulsified forms. Some existing hydroalcoholic gels as delivery systems are structured with colloidal silicates, polyacrylic acid, emulsifiers or surfactants. Even though they may have a clear appearance, the manufacture of many of these hydroalcoholic gels requires heating.

An effective delivery system for hydrophobic substrates should allow high oil incorporation to maximize the content of oil soluble active compounds. An effective topical delivery system should also not cause any skin irritation upon chronic, repeated application. Currently, most carrier bases used as pharmaceuticals do not feature penetration enhancers to help deliver active ingredients across the skin barrier and there are no existing products that can self-emulsify into a cream upon the addition of oils.

### SUMMARY OF PARTICULAR EMBODIMENTS

It will be appreciated by those skilled in the art that other variations of the embodiments described below may also be practiced without departing from the scope of the invention. Further note, these embodiments, and other embodiments of the present invention will become more fully apparent from a review of the description and claims which follow.

In one embodiment, the present disclosure relates to a composition of a hydroalcoholic gel and method of manufacturing same. In one particular embodiment, this hydroalcoholic gel self-emulsifies into a cream when mixed with oils. In one further particular embodiment, this hydroalcoholic gel can be used as a carrier for topical or transdermal delivery of pharmaceutical active ingredients and bioactive ingredients.

In one embodiment, the present disclosure includes the composition of a hydroalcoholic gel and method of manufacture. In one particular embodiment, this hydroalcoholic gel contains water, lower alcohol, gelling agents, thickening agents, emulsifiers, and skin conditioning agents. In one embodiment, this hydroalcoholic gel also contains penetration enhancers to help topical delivery of hydrophobic and/or hydrophilic substrates. In one embodiment of a method of manufacture, this hydroalcoholic gel is manufactured through a cold process. In one particular embodiment, this hydroalcoholic gel has a transparent or semi-transparent appearance and it will become opaque with the addition of oil, indicating homogeneous mixing. The emulsified cream form of the composition is stable for at least three months. In another particular embodiment, the composition can be used as a carrier for topical delivery of pharmaceutical active ingredients and bioactive ingredients, and has shown superior topical delivery efficacy of hydrophobic and hydrophilic substances. In one embodiment, the emulsified hydroalcoholic gel also allows convenient application for end users as comparing to applying a highly-mobile oil or solution to the skin surface.

### DETAILED DESCRIPTION OF EXAMPLE EMBODIMENTS

In this respect, before explaining at least one embodiment of the invention in detail, it is to be understood that the invention is not limited in its application to the details of construction and the arrangements of the components outlined in the following description or illustrated in the drawings. The invention is capable of other embodiments and of being practiced and carried out in various ways. Also, it is to be understood that the phraseology and terminology employed herein are for the purpose of description and should not be regarded as limiting. In particular, all terms used herein are used in accordance with their ordinary meanings unless the context or definition clearly indicates otherwise. Also, unless indicated otherwise except within the claims the use of “or” includes “and” and vice-versa. Non-limiting terms are not to be construed as limiting unless expressly stated or the context clearly indicates otherwise (for example, “including”, “having”, “characterized by” and “comprising” typically indicate “including without limitation”). Singular forms included in the claims such as “a”, “an” and “the” include the plural reference unless expressly stated or the context clearly indicates otherwise. Further, the stated features and/or configurations or embodiments thereof the suggested intent may be applied as seen fit to certain operating conditions or environments by one experienced in the field of art.

The present disclosure provides oral care compositions, methods of manufacture for the composition, and methods of administration or application to, or use with, a human or other animal subjects. In one embodiment, a hydroalcoholic gel composition is provided, containing water, lower alcohol, gelling agents, thickening agents, emulsifiers, and skin conditioning agents. This hydroalcoholic gel composition is intended to be used as a carrier for hydrophobic and hydrophilic pharmaceutical and cosmeceutical bioactive substrates, added as is or added when dispersed in oil. The mixed hydroalcoholic gel is intended to be applied to intact skin surface. Materials to be used in these compositions should be food, pharmaceutically and/or cosmetically acceptable.

In this disclosure, the term “hydroalcoholic gel” is intended to refer to compositions with water and lower alcohols as the liquid medium of a gel.

In this disclosure, the term “carrier” is intended to refer to compositions in which bioactive substrates are dispersed.

In this disclosure, the term “topical delivery” is intended to refer to the delivery of bioactive substrates through intact skin tissue across the skin barrier, namely the stratum corneum.

In this disclosure, the term “cold process” is intended to refer to a process of manufacture that does not require external source of heating, however, heat might be generated by physicochemical reactions between substrates in the compositions.

In this disclosure, the term “self-emulsify” is intended to refer to the process whereby oil is added into the hydroalcoholic gel, a homogeneous emulsion can be achieved by stirring the substrate and/or shaking the container; and no additional mixing devices or steps are required to achieve the homogeneous emulsion.

In this disclosure, the term “alcohol” is intended to refer to lower alcohols that contain 1-6 carbons, preferably ethyl alcohol, denatured ethyl alcohol and the like. The accumulative concentration of alcohol in the present disclosure may be at 5% to 95% by weight.

In this disclosure, the term “gelling agents” is intended to refer to substrates that are dispersed and/or dissolved in water-alcohol medium to form a weakly cohesive internal structure and have a semi-solid texture. Acceptable gelling agents to be used can be, but are not limited to, carbomers, (block) copolymers carrageenans, agar, natural gums and their modifications such as alginates, guar gum, karaya gum, xanthan, gum Arabic and tragacanth, colloidal magnesium aluminum silicate, colloidal silica, hyaluronic acids and modifications and the like. One or more gelling agents may present in a total content of 0.01% to 5% by weight.

In this disclosure, “thickeners” is intended to refer to substrates that can increase the viscosity of the hydroalcoholic gel. Acceptable thickeners to be used can be, but not limited to, carbomers, (block) copolymers, carrageenans, cellulosic polymers such as hydroxyethylcellulose, carboxymethylcellulose, hydroxypropyl methylcellulose and salts, agar, natural gums and modified natural gums such as alginates, guar gum, karaya gum, xanthan, gum Arabic and tragacanth, colloidal magnesium aluminum silicate, colloidal silica, hydrogenated oils, fatty alcohols, fatty acids, fatty acid salts, fatty esters, polyethylene glycols (PEGs) and derivatives such as PEG-hydrogenated caster oils, polyglycerol fatty esters, hyaluronic acids and modifications, and the like. One or more thickening agents may present in a total content of 0.01% to 5% by weight.

In this disclosure, the term “emulsifying agents” is intended to refer to substrates that will help with the incorporation of oil into the hydroalcoholic gel and allow the self-emulsification process. Acceptable emulsifying agents to be used can be, but are not limited to, polysorbates, fatty acid salts, fatty alcohols, fatty acid esters, polyethylene glycols and derivatives, polar lipids, (block) copolymers, (poly) acrylic acids, polyglycerol fatty esters, betaines, sugar esters, and the like. One or more emulsifying agents may present in a total content of 0.01% to 10% by weight.

Penetration enhances (PEs) are substrates that could help bioactive ingredients to be delivered across the skin barrier, and improve the mass penetrated into the skin. Typical ways of action of PEs are skin barrier disruption or complex formation with active ingredients. Acceptable PEs to be used in the current invention can be, but not limited to, lower alcohol such as ethyl alcohol and denatured ethyl alcohol, glycols such as ethoxydiglycol, dimethyl sulfoxide, isopropyl palmitate, isopropyl myristate, surfactants such as sodium lauryl sulfate, sodium laureth sulfate, polysorbates, sugar esters, fatty acid salts, and the like. One or more PEs may present in a total content of 0.1% to 95% by weight.

Skin conditioning agents in the present disclosure may function as emollients, humectants and occlusive. Acceptable skin conditioning agents to be used in the current invention can be, but are not limited to, oils, fatty alcohols, glycols, fatty acids, fatty alcohols, fatty acid esters, glycerin, glycols, polyethylene glycols (PEGs) and derivatives, silicones, polysaccharides and modified polysaccharides including hyaluronic acid and its modifications, natural gums and their modifications and the like. Skin conditioning agents may present in a total content of 0.1 to 40% by weight.

Optional ingredients, such as, without limit to, colouring agents, fragrances, preservatives, chelating agents, pH modifiers and antioxidants can also be included into the compositions described herein.

In one embodiment, the composition of the hydroalcoholic gel may hold up to 100% of its weight of oil, from animal, plant, and mineral origins, through a self-emulsification process. The emulsified hydroalcoholic gel in the form of a lotion or cream, may contain up to 50% of its weight of oil. Once fully emulsified, the hydroalcoholic gel changes its appearance from a clear to semi-clear gel into an opaque lotion or cream. The emulsified hydroalcoholic gel is stable at room temperature for at least three months.

Hydrophobic substrates can be dissolved firstly in an oil, and the oil can be added into the hydroalcoholic gel. Hydrophilic substrates can be incorporated directly into the hydroalcoholic gel, with or without an oil.

Selected compositions of hydroalcoholic gel mixed with oils have been sent to a third party laboratory for testing. The compositions do not cause skin irritations or other adverse effects after long-term repeated applications to intact skin.

The application of the hydroalcoholic gel and its emulsion is non-occlusive. The hydroalcoholic gel can be applied directly to intact skin surfaces. The hydroalcoholic gel can be applied to intact skin surface as is, or emulsified with oils, if only hydrophilic substrates are incorporated into the carrier. The hydroalcoholic gel can be applied to intact skin surface once emulsified with oil which has hydrophobic substrates dissolved within. A thin and even layer of the (emulsified) hydroalcoholic gel may be rubbed to intact skin surface until the preparation disappears via absorption.

In order to obtain a homogeneous cream or lotion, oil up to 100% weight of the hydroalcoholic gel can be added into the hydroalcoholic gel. The gel-oil mixture can be stirred with a spoon, or be shaken in a closed container, to form a homogeneous emulsion through a self-emulsification process.

The hydroalcoholic gel composition can be manufactured based on the following example method.

The hydroalcoholic gel composition of the present invention may be prepared by suitably mixing the ingredients in sequence. In one example method, gelling agents and thickeners are firstly dispersed into 50% to 100% of water, and mixed to allow the substrates to fully dissolve or hydrate. Alcohols are then added into the vessel. Emulsifying agents, other penetration enhancers, skin conditioning agents, and optional ingredients can then be added into the vessel and the composition is mixed until thoroughly until homogeneous. Hydrophilic bioactive substrates can be added into the vessel and the composition is mixed thoroughly until homogeneous.

### EXAMPLES

### Example 1—A Formulation of Hydroalcoholic Gel. Ingredients are Listed by % Weight

### Water 65.9%

Ethyl alcohol 15%

### Ethoxydiglycol 10%

Propylene glycol 5%
Sodium lauryl sulfate 3%

### Xanthan gum 1%

Sodium benzoate 0.1%

### Example 2—A Method of Making the Composition of Hydroalcoholic Gel Shown in Example 1

1. Fill a mixing vessel with water;
2. Disperse xanthan gum in the vessel and allow it to fully hydrate; and
3. Disperse ethyl alcohol, ethoxydiglycol, propylene glycol, sodium lauryl sulfate and sodium benzoate into the vessel and mix until homogeneous.

### Example 3—Hydroalcoholic Gel as a Carrier to Hydrophobic Substrates

A composition of the present hydroalcoholic gel has been tested to determine its efficacy as a carrier for topical delivery of a hydrophobic substrate, such as THC. THC has been dispersed in a plant oil. The THC-containing oil has been mixed with the hydroalcoholic gel composition at 50% of its volume, yielding a total oil content of 33% volume of the emulsified cream. The THC content in the emulsified hydroalcoholic gel is 0.37% wt. 1×1 ml aliquot was given to healthy adults aged between 25-55 years old who had not used any cannabis products for one month. Each person applied their 1 mL of the topical cannabis cream to the inner wrist and neck. All volunteers experienced a psychoactive effect after topical application of the emulsified gel. Urine samples were collected two hours after application of the cream, and these urine samples were tested with a urine alcohol test strip and a urine THC test strip. The urine samples collected from all volunteers were negative for alcohol while 50% of them showed positive results for THC. The literature has shown that typically, topical application of THC containing products is not able to cause a positive response in blood or urine (Hess, Krämer, and Madea, 2017); therefore the composition of the present invention is an efficient carrier to topically deliver THC.

### Example 4—Hydroalcoholic Gel as a Carrier to Hydrophilic Substrates

A composition of the present hydroalcoholic gel has been tested to determine its efficacy as a carrier for topical delivery of a hydrophilic substrate. Niacin (5% wt.) and arginine (3% wt.) have been dispersed in a hydroalcoholic gel composition. Niacin typically causes increased blood flow after oral ingestion, resulting in observable skin redness. The active-containing hydroalcoholic gel, an active-containing conventional carrier cream, and a negative control hydroalcoholic gel that does not contain any niacin or arginine were given to health adults between 24-60 years old. 1 mL aliquot of each was given to the volunteers to apply to their forearm. Skin rash or redness were only observed from skin area rubbed with active-containing hydroalcoholic gel, indicating topical delivery of niacin across the skin barrier.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, functions, operations, or steps, any of these embodiments may include any modification, combination or permutation of any of the components, elements, functions, operations, or steps described or illustrated anywhere herein that a person having ordinary skill in the art would comprehend. All such modifications, combinations and permutations are believed to be within the sphere and scope of the invention as defined by the claims appended hereto.

What is claimed is:

1. A hydroalcoholic gel composition consisting essentially of water, a lower alcohol, a gelling agent, a thickener, an emulsifier, a penetration enhancer, and a skin conditioning agent.

2. The hydroalcoholic gel composition according to claim 1, containing about 5% to 95% by weight of lower alcohol.

3. The hydroalcoholic gel composition according to claim 1, wherein the lower alcohol contains between 1 and 6 carbon atoms.

4. A hydroalcoholic gel composition according to claim 1, containing about 0.01% to 5% by weight of gelling agents.

5. A hydroalcoholic gel composition according to claim 1, containing about 0.01% to 5% by weight of thickeners.

6. A hydroalcoholic gel composition according to claim 1, containing about 0.01% to 10% by weight of emulsifying agents.

7. A hydroalcoholic gel composition according to claim 1, containing about 0.01% to 95% by weight of penetration enhancers.

8. A hydroalcoholic gel composition according to claim 1, containing about 0.1% to 40% by weight of skin conditioning agents.

9. The hydroalcoholic gel composition according to claim 1, for use as a carrier for topical delivery of hydrophilic substrates.

10. The hydroalcoholic gel composition according to claim 1, for use in enhancing the efficacy of topical delivery of hydrophilic substrates.

11. The hydroalcoholic gel composition according to claim 1, for use as a carrier for topical delivery of hydrophobic substrates.

12. The hydroalcoholic gel composition according to claim 1, for use in enhancing the efficacy of topical delivery of hydrophobic substrates.

13. A hydroalcoholic gel composition consisting essentially of by weight of the total weight of the composition: about 5% to 95% by weight lower alcohol, about 0.01% to 5% by weight gelling agents, about 0.01% to 5% by weight thickeners, about 0.01% to 10% by weight emulsifying agents, about 0.01% to 95% by weight penetration enhancers, about 0.1% to 40% by weight of skin conditioning agents, and water q.s. to 100%.

14. The hydroalcoholic gel composition according to claim 13, for use as a carrier for topical delivery of hydrophilic substrates.

15. The hydroalcoholic gel composition according to claim 13, for use in enhancing the efficacy of topical delivery of hydrophilic substrates.

16. The hydroalcoholic gel composition according to claim 13, for use as a carrier for topical delivery of hydrophobic substrates.

17. The hydroalcoholic gel composition according to claim 13, for use in enhancing the efficacy of topical delivery of hydrophobic substrates.

18. A method of enhancing the dermal or topical delivery of a biological active comprising administering a biologically effective amount of a composition according to claim 13 to the skin or mucosa of a human or animal.

19. The method of claim 18 wherein the biological active is a hydrophilic substrate.

20. The method of claim 18 wherein the biological active is a hydrophobic substrate.
